(19) United States
(12) Patent Application Publication
(10) Pub. No.: US 2025/0283050 A1
(43) Pub. Date: Sep. 11, 2025

(54) METHOD OF CANCER TREATMENT

(71) Applicant: Zhenglun Zhu, Newton, MA (US)

(72) Inventor: Zhenglun Zhu, Newton, MA (US)

(21) Appl. No.: 18/700,844

(22) PCT Filed: Oct. 14, 2022
(86) PCT No.: PCT/US2022/046781

§ 371 Date: Apr. 12, 2024

Related U.S. Application Data
(60) Provisional application No. 63/256,391, filed on Oct. 15, 2021.

(51) Int. Cl. C12N 5/09; C07K 16/30; C12N 5/071; G01N 33/50; G01N 33/543

(52) U.S. Cl. CPC C12N 5/0693; C07K 16/30; C12N 5/0688; G01N 33/5011; G01N 33/5082; G01N 33/54366; C12N 2502/1157; C12N 2503/02; G01N 2500/10

(57) ABSTRACT

The invention described herein provides methods for an ex vivo culture model or en bloc culture of solid tumor/cancer and used thereof, for fast, efficient, and accurate assessment of potential therapeutic methods to treat cancer.

### REFERENCE TO RELATED APPLICATION

This application claims priority and the benefit of the filing date of U.S. Provisional Patent Application No. 63/256,391, filed on Oct. 15, 2021, the entire contents of which are incorporated herein by reference.

### BACKGROUND OF THE INVENTION

Lung cancer is the leading cause of cancer-related death worldwide, with a median survival of approximately 1 year, and an overall 5-year survival of less than 20%. The 5-year survival rate declines precipitously to 5% for patients with distant metastases, which account for more than 55% of newly diagnosed cases. Pathologically, about 85% of lung cancers are non-small cell lung cancer (NSCLC), of which about 70% are adenocarcinoma (L-ADCA) and 20% are squamous cell carcinoma (L-SCCA).

Unlike the dismal effects of adjuvant chemotherapy, recent FDA-approved immune check point inhibitor (ICIs) treatment have demonstrated unprecedent success against NSCLC. Nonetheless, overall, only 20% NSCLC patients respond to ICIs. Moreover, the majority of the responders eventually relapse and succumb to the disease during the long-term 5-year follow-up. Therefore, improvement to ICI-based lung cancer treatment, partly based on mechanistic understanding and insight of ICI resistance, is urgently needed for developing further strategies to improve ICI efficacy against NSCLC.

CD8⁺ cytotoxic T lymphocytes (CTL) are key immune effector cells in tumor immunology. CD8⁺ T cell functions are modulated by co-stimulatory and co-inhibitory molecules to ensure adequate balance between immune reaction against foreign/tumor antigens and safeguard against excessive auto-immune inflammatory response. Interaction of co-inhibitory check point receptors with their ligands on tumor and stromal cells accounts for inhibition/exhaustion of tumor associated CD8⁺ T cells and the evasion of tumor cells from immune destruction during immunopathogenesis of NSCLC. ICIs, such as the PD-1 antibody and the CTLA-4 antibodies function, in part, by blocking CD8 inhibition, therefore, reinvigorate CD8⁺ CTL. Inadequate antigen presentation and immune suppression of CD8⁺ CTL within tumor microenvironment (TME) have been proposed as the two possible reasons for ICI treatment failure. However, with limited knowledge on immune cell profiling and the lack of a platform to dissect the functional interaction of ICIs and immune cells within the TME, targeting the TME to enhance the efficacy of ICI against NSCLC remains a theoretical rationale, awaiting biochemical data support and clinical trial approval.

### SUMMARY OF THE INVENTION

Tumor antigen recognition and CTL activation is at the forefront of cancer immunotherapy. While systemic stimulation of CD8⁺ CTL by ICIs have led to unprecedent success in cancer immunotherapy, the ICI efficacy against cancer (such as NSCLC) remains limited, and ICI induced irAE can be severe. Immune suppression of TME has been recognized as a central player in ICI resistance, but efforts in targeting TME have yet to produce impactful results in treatment of NSCLC and other solid tumors, in part, due to limited understanding of the composition and regulatory mechanisms of TME.

Thus one aspect of the invention provides an ex vivo culture model or en bloc culture of solid tumor/cancer (such as lung cancer including NSCLC), for testing the efficacy a therapeutic method on treating the tumor/cancer, comprising freshly isolated tissue sample of the solid tumor/cancer cultured in a suitable mammalian tissue culture medium, wherein the freshly isolated tissue sample is reduced in size to about 1-10 mm (e.g., 2-8 mm, 3-6 mm, about 5 mm) in the largest dimension.

Another aspect of the invention provides a method to assess the efficacy or effectiveness of a therapy in order to treat a solid tumor/cancer in a subject having said solid tumor/cancer, the method comprising contacting a therapeutic agent for the therapy with the subject ex vivo culture model or en bloc culture, for said solid tumor/cancer isolated from said subject, and identifying a favorable outcome after a sufficient period of time, wherein the favorable outcome indicates that said subject is suitable to be treated by said therapy, and/or wherein the method further comprises selecting said subject for treatment by said therapy upon observation of a favorable outcome, wherein the favorable outcome: (1) with respect to the therapeutic agent that comprises a chemotherapy agent (such as the chemotherapeutic agent at a sub-therapeutic dose insufficient to treat said solid tumor/cancer), comprises elevated/increased Hom-1 expression in tumor-associated macrophages (TAM) in said ex vivo culture model or en bloc culture (as compared to Hom-1 expression without contacting the therapeutic agent); or, (2) with respect to the therapeutic agent that comprises an immune checkpoint inhibitor (ICI), comprises cytocidal effect on tumor/cancer cells; activation of cytotoxic T cells (CTL) or CD8⁺ T cells; elevated expression and/or secretion of pro-inflammatory cytokines (such as IL-1β, IL-8, IL-12B and TNF-α) and/or reduction in expression of immune suppressive cytokines (such as the IL-4, IL-10, IL13 and TGF-β); and/or death of tumor cells in the tissue cultures.

Another aspect of the invention provides a method of treating a cancer, such as solid a cancer/tumor (e.g., lung cancer including NSCLC), the method comprising administering a therapy to a subject having said cancer, wherein the subject has been validated to respond to treatment by said therapy according to a favorable outcome in any one of the subject method to assess the efficacy or effectiveness of the therapy for treating said solid tumor/cancer using the ex vivo culture model or en bloc culture of said solid tumor/cancer.

Another aspect of the invention provides a method to enhance immune checkpoint inhibitor (ICI)-mediated therapy of a cancer (e.g., treatment of NSCLC) in a subject, the method comprising promoting tumor-specific activation of CD8⁺ T cells in tumor microenvironment (TME) of the cancer through Hom-1 activation in, or Hom-1 mediated activation of, tumor-associated macrophages (TAMs).

Another aspect of the invention provides a method to break resistance to immune checkpoint inhibitor (ICI)-mediated therapy of a cancer (e.g., treatment of NSCLC) in a subject, the method comprising promoting tumor-specific activation of CD8⁺ T cells in tumor microenvironment (TME) of the cancer through Hom-1 activation in, or Hom-1 mediated activation of, tumor-associated macrophages (TAMs).

Another aspect of the invention provides a method to enhance efficacy of chemotherapeutic agent therapy of a cancer (e.g., treatment of colorectal cancer) in a subject, the method comprising promoting tumor-specific activation of CD8⁺ T cells in tumor microenvironment (TME) of the cancer through Hom-1 activation in, or Hom-1 mediated activation of, tumor-associated macrophages (TAMs).

Another aspect of the invention provides a method to break resistance to chemotherapeutic resistance of a cancer (e.g., treatment of colorectal cancer) in a subject, the method comprising promoting tumor-specific activation of CD8⁺ T cells in tumor microenvironment (TME) of the cancer through Hom-1 activation in, or Hom-1 mediated activation of, tumor-associated macrophages (TAMs).

Another aspect of the invention provides a method to select an effective dosage of a therapy (e.g., chemotherapeutic agent, targeted therapy, immunotherapeutic treatment, radiation therapy, or combinations thereof) for treating a cancer (e.g., lung cancer or colorectal cancer) in a subject, the method comprising determining Hom-1 activation in, or Hom-1 mediated activation of, tumor-associated macrophages (TAMs) of said cancer (e.g., ex-vivo, in vivo, or both), upon contacting the cancer with said therapy, wherein the minimum effective dosage of said therapy that leads to Hom-1 activation, or a higher dosage, is selected to be the effective dosage.

Another aspect of the invention provides a method to select an effective dosage of a therapy (e.g., chemotherapeutic agent, targeted therapy, immunotherapeutic treatment, radiation therapy, or combinations thereof) for treating a cancer (e.g., lung cancer or colorectal cancer) in a subject, the method comprising contacting the cancer with said therapy to identify the minimum effective dosage of said therapy that promotes tumor-specific activation of CD8⁺ T cells in tumor microenvironment (TME) of said cancer through Hom-1 activation in, or Hom-1 mediated activation of, tumor-associated macrophages (TAMs).

Any embodiments described herein, including those only in the examples or claims, can be combined with any other one or more embodiments of the invention, unless expressly disclaimed or are improper.

### DETAILED DESCRIPTION OF THE INVENTION

### 1. Overview

The invention described herein is partly based on the characterization of the molecular and cellular compositions of NSCLC-TME, and the analysis of the functional interaction of ICI and immune cells within NSCLC-TME.

Data presented herein showed that NSCLC-TME contains abundant CD8⁺ T cells which exhibit an exhausted phenotype. Application of PD-1 antibody activates CD 8⁺ T cells, but exerts limited effects on the immune landscape of NSCLC-TME. Further, expression of the homeobox gene Hom-1 as the master regulator of macrophage plasticity and immune polarity is significantly down-regulated in tumor associated macrophages (TAMs) of NSCLC.

As used herein, “Hom-1” is used interchangeably with “VentX” (as the term is used in the priority application U.S. Ser. No. 63/256,391).

Meanwhile, restoration of Hom-1 expression in NSCLC-TAMs transforms the immune landscape of NSCLC-TME from immune suppression to activation, and that Hom-1-modulated-TAMs promotes tumoricidal effects of PD-1 antibody on NSCLC by 4-5 folds, and promotes tumoricidal effects of chemotherapeutic agents able to activate Hom-1 expression in TAMs/macrophages/monocytes (such as chemotherapeutic agents able to up-regulate NF-KB-mediated Hom-1 expression, e.g., Doxorubicin (DOX)) by about 10 folds, all without leading to cytotoxic effects on normal tissues. In certain embodiments, the chemotherapeutic agent is able to activate Hom-1 expression at a concentration below, e.g., far below the concentration required for cytotoxicity—e.g., a subtherapeutic dose not by itself sufficient to treat cancer via its cytotoxicity. As such, immunotherapy and chemotherapy can be applied at non-cytotoxic or suboptimal or subtherapeutic dosage to achieve its therapeutic goal.

Furthermore, Hom-1-modulated-TAMs were able to promote the efficacy of PD-1-antibody against NSCLC tumorigenesis in pre-clinical NSG-PDX models of individual primary human NSCLC.

Thus, the invention described herein provides a method to enhance immune checkpoint inhibitor (ICI)-mediated therapy or chemotherapy of a cancer (e.g., treatment of NSCLC) in a subject, the method comprising promoting tumor-specific activation of CD8⁺ T cells in tumor microenvironment (TME) of the cancer through Hom-1 activation in, or Hom-1 mediated activation of, tumor-associated macrophages (TAMs).

In a related aspect, the invention described herein provides a method to break resistance to immune checkpoint inhibitor (ICI)-mediated therapy of a cancer (e.g., treatment of NSCLC) in a subject, the method comprising promoting tumor-specific activation of CD8⁺ T cells in tumor microenvironment (TME) of the cancer through Hom-1 activation in, or Hom-1 mediated activation of, tumor-associated macrophages (TAMs).

In certain embodiments, Hom-1 activation promotes phagocytosis of cancer cells by said TAMs.

In certain embodiments, the method comprises: (1) promoting/inducing/enhancing Hom-1 activation in, or Hom-1 mediated activation of, tumor-associated macrophages (TAMs), such as by contacting said TAMs with a sub-therapeutic dose of a chemotherapeutic agent (such as Doxorubicin) that induce the expression of Hom-1, wherein said sub-therapeutic dose of the chemotherapeutic agent is by itself insufficient to treat said cancer alone; (2) contacting cancer cells with said TAMs with increased Hom-1 activity or expression in (1) to enhance phagocytosis of said cancer cells, and (3) contacting CD8⁺ T cells with said TAMs in (2) to activate said CD8⁺ T cells.

In certain embodiments, the method further comprises (4) expanding activated CD8⁺ T cells ex vivo or in vitro.

In certain embodiments, the method is an ex vivo method. For example, TAMs/monocytes/macrophages can be isolated from patient tumor/cancer sample and optionally expanded/cultured in vitro under standard culture conditions. Such TAMs monocytes/macrophages can then be modified/activated or induced to express Hom-1. TAMs monocytes/macrophages with increased Hom-1 expression can then be contacted with cancer cells to permit phagocytosis of the cancer cells. Subsequently, such TAMs monocytes/macrophages can be contacted with CD8⁺ T cells, such as CD8⁺ T cells isolated from the same patient with the cancer, or TIL, to activate the CD8⁺ T cells. CD8⁺ T cells so activated may optionally be expanded under standard tissue culturing conditions, before they are administered back into the patient from which the CD8⁺ T cells are isolated.

In other embodiments, the method is an in vivo method. For example, a lose dose (e.g., a subtherapeutically effective dose that is insufficient by itself to treat cancer or causing tumoricidal effect) may be administered to a patient in need to treatment, such that TAMs monocytes/macrophages is exposed to such lose dose chemotherapeutic agent that elevates Hom-1 expression, for example, through stimulation of the NF-KB signaling.

In certain embodiments, said ICI-mediated therapy comprises administering an antibody or antigen-binding fragment thereof specific for an inhibitory immune checkpoint target, such as PD-1, PD-L1, PD-L2, CTLA-4/CD152, A2AR, B7-H3/CD276, B7-H4/VTCN1, BTLA/CD272, IDO, KIR, LAG3, NOX2, TIM-3, VISTA, galectin-9, SIGLEC7/CD328, or SIGLEC9.

In certain embodiments, the antibody or antigen-binding fragment thereof is specific for PD-1, PD-L1, or PD-L2. In certain embodiments, the antibody or antigen-binding fragment thereof is specific for PD-1.

In certain embodiments, the TAM has down-regulated expression of Hom-1 (e.g., in the TME) prior to said Hom-1 activation.

In certain embodiments, the cancer is lung cancer, such as NSCLC.

Another aspect of the invention provides an ex vivo culture model or en bloc culture of solid tumor/cancer, such as NSCLC, for testing the efficacy a therapeutic method on treating the tumor/cancer, comprising freshly isolated (optionally having been frozen) tissue sample of the solid tumor/cancer cultured in a suitable mammalian tissue culture medium, wherein the freshly isolated tissue sample is reduced in size to about 1-10 mm (e.g., 2-8 mm, 3-6 mm, about 5 mm) in the largest dimension.

In certain embodiments, the mammalian tissue culture medium is formulated for suspension cell culture, such as RPMI 1640 medium or RPMI 1640 complete medium.

In certain embodiments, the mammalian tissue culture medium is supplemented with 2-10% FBS; optionally, the mammalian tissue culture medium is further supplemented with an antibiotics, such as 1-2.5% antibiotic-antimycotic solution.

In certain embodiments, the freshly isolated tissue sample of the solid tumor/cancer is cultured in a the suitable mammalian tissue culture medium in a 24-well tissue culture plate.

In certain embodiments, the freshly isolated tissue sample is first washed in a buffer, such as 1×PBS buffer with antibiotics, prior to reduction in size.

Another aspect of the invention provides a method to assess the efficacy or effectiveness of a therapy in order to treat a solid tumor/cancer in a subject having said solid tumor/cancer, the method comprising contacting a therapeutic agent (or combination of agents) for the therapy with the subject ex vivo culture model or en bloc culture, for said solid tumor/cancer isolated from said subject, and identifying a favorable outcome after a sufficient period of time, wherein a favorable outcome indicates that said subject is suitable to be treated by said therapy, and/or wherein the method further comprises selecting said subject for treatment by said therapy upon observation of a favorable outcome, wherein the favorable outcome: (1) with respect to the therapeutic agent that comprises a chemotherapy agent (such as the chemotherapeutic agent at a sub-therapeutic dose insufficient to treat said solid tumor/cancer), comprises elevated/increased Hom-1 expression in tumor-associated macrophages (TAM) in said ex vivo culture model or en bloc culture (as compared to Hom-1 expression without contacting the therapeutic agent); or, (2) with respect to the therapeutic agent that comprises an immune checkpoint inhibitor (ICI), comprises cytocidal effect on tumor/cancer cells; activation of cytotoxic T cells (CTL) or CD8⁺ T cells; and/or elevated expression and/or secretion of pro-inflammatory cytokines (such as IL-1β, IL-8, IL-12B and TNF-α) and/or reduction in expression of immune suppressive cytokines (such as the IL-4, IL-10, IL13 and TGF-β).

It should be noted that the method of the invention is not limited to chemotherapeutic agent or ICI-mediated therapy. Therapeutic interventions such as radiotherapy, Car-T-based immunotherapy etc., can also benefit from the method of the invention, so long as such therapies may lead to Hom-1 activation in, or Hom-1 mediated activation of, tumor-associated macrophages (TAMs).

Thus, in certain embodiments, the favorable outcome comprises Hom-1 activation in, or Hom-1 mediated activation of, tumor-associated macrophages (TAMs).

In certain embodiments, the solid tumor/cancer is lung cancer, such as NSCLC.

In certain embodiments, the therapy is chemotherapy, optionally, the therapeutic agent comprises a chemotherapeutic agent, such as Doxorubicin (DOX).

In certain embodiments, the therapy is immunotherapy, optionally, the therapeutic agent comprises an immune checkpoint inhibitor (ICI).

In other embodiments, the immunotherapy comprises an antigen based approach, such as therapy with CAR-T cells, or therapy comprising tumor antigen stimulation.

In certain embodiments, the ICI comprises an antibody or antigen-binding fragment thereof. In certain embodiments, the antibody or antigen-binding fragment thereof is specific for an inhibitory immune checkpoint target, such as PD-1, PD-L1, PD-L2, CTLA-4/CD152, A2AR, B7-H3/CD276, B7-H4/VTCN1, BTLA/CD272, IDO, KIR, LAG3, NOX2, TIM-3, VISTA, galectin-9, SIGLEC7/CD328, or SIGLEC9.

In certain embodiments, the antibody or antigen-binding fragment thereof is specific for PD-1, PD-L1, or PD-L2. In certain embodiments, the antibody or antigen-binding fragment thereof is specific for PD-1, such as 1 μg/mL of Pembrolizumab.

In certain embodiments, the ex vivo culture model or en bloc culture of said solid tumor/cancer is contacted by said therapeutic agent for at least 1-2 days.

In certain embodiments, the method further comprises contacting the ex vivo culture model or en bloc culture of the solid tumor/cancer with a second therapeutic agent.

In certain embodiments, the second therapeutic agent comprises a macrophage or a monocyte having elevated/increased Hom-1 expression (e.g., induced or modified to express Hom-1).

In certain embodiments, the second therapeutic agent comprises a immune therapeutic agent, chemotherapeutic agents, targeted therapy agents, a radiation methods/agents.

In certain embodiments, the macrophage or a monocyte is an autologous macrophage or a monocyte from the same subject from which the solid tumor/cancer is isolated.

In certain embodiments, the macrophage or a monocyte is modified to express elevated levels of Hom-1.

In certain embodiments, the macrophage or a monocyte is induced to express Hom-1.

In certain embodiments, the macrophage or a monocyte is modified to express Hom-1 by introducing into the macrophage or monocytes a heterologous construct encoding Hom-1.

In certain embodiments, the heterologous construct encoding Hom-1 comprises a plasmid encoding Hom-1.

In certain embodiments, the heterologous construct encoding Hom-1 comprises a nanoparticle encompassing an mRNA encoding Hom-1.

In certain embodiments, the heterologous construct encoding Hom-1 comprises a viral vector (such as an AAV vector) encoding Hom-1.

In certain embodiments, the method comprises introducing into the macrophage or monocyte heterologous Hom-1 protein.

In certain embodiments, the sufficient period of time comprises about 3-6 days, such as 3, 4, 5, 6, 7, or 8 days culturing at 37° C. and under 5% CO₂.

In certain embodiments, the outcome is determined by isolating single cells from the ex vivo culture model or en bloc culture of said solid tumor/cancer.

In certain embodiments, determining the favorable outcome comprises: assessing the viability or death of cancer cells, assessing the number and/or function of CD8⁺ and/or CD4⁺ lymphocytes (optionally including the number of T_{reg}) and/or numbers/functions of macrophages (including TAMs) in the ex vivo culture model or en bloc culture, the expression of cell surface check point inhibitors (such as PD-1 and CTLA-4), the expression of effector molecules (such as the IFN-γ and Granzyme B), the M1-or M2-like phenotype of the TAMs, the expression of immune suppressive cytokines (such as the IL-4, IL-10, IL13 and TGF-β), and/or the expression of the pro-inflammatory cytokines (such as IL-1β, IL-8, IL-12B and TNF-α).

In certain embodiments, determining the favorable outcome comprises FACS analysis of isolated single cells from the ex vivo culture model or en bloc culture, and/or ELISA analysis of culture supernatants from the ex vivo culture model or en bloc culture (e.g., ELISA analysis of IL-2 and IFN-γ expression).

In certain embodiments, determining the favorable outcome comprises determining cell surface expression of CD68 and CD206 on TAMs (e.g., via FACS), and/or Hom-1 expression level (e.g., via qRT-PCR analysis).

In certain embodiments, determining the favorable outcome comprises determining the percentage of T_{reg} cells, e.g., by FACS analysis of the percentage changes of the CD4⁺CD25⁺FoxP3⁺ cells.

In certain embodiments, determining the favorable outcome comprises determining the change or enhancement of CD8⁺ T cell activation upon contact by the therapeutic agent (e.g., an ICI antibody, such as anti-PD-1 antibody).

In certain embodiments, determining the favorable outcome comprises determining the percentage of surviving or remaining cancer cells, and/or viability of cancer cells.

In certain embodiments, determining the favorable outcome comprises determining the percentage of surviving or remaining cancer cells, and/or viability of cancer cells. For example, this could include immunohistochemical analysis of markers whose expression levels are elevated in tumors.

In certain embodiments, the method comprises comparing the favorable outcome with that of a control outcome obtained by contacting a control therapeutic agent for the therapy with the ex vivo culture model or en bloc culture of said solid tumor/cancer.

In certain embodiments, the therapeutic agent is an antibody or antigen-binding fragment thereof, and the control therapeutic agent is an isotype matched control antibody or antigen-binding fragment thereof (such as IgG1 or IgG4).

Another aspect of the invention provides a method of treating a cancer, such as a solid cancer/tumor (e.g., lung cancer including NSCLC), the method comprising administering a therapy to a subject having said cancer, wherein the subject has been validated to respond to treatment by said therapy according to a favorable outcome in the method to assess the efficacy or effectiveness of the therapy for treating said solid tumor/cancer using the ex vivo culture model or en bloc culture of said solid tumor/cancer.

In certain embodiments, the therapy is chemotherapy comprising administering chemotherapeutic agents either alone or in combination to said subject.

In certain embodiments, the therapy is chemotherapy comprising administering Doxorubicin (DOX) to said subject.

In certain embodiments, the therapy is immune therapy comprising administering an ICI to said subject.

In certain embodiments, the ICI is an antibody or antigen-binding fragment thereof specific for an inhibitory immune checkpoint target, such as PD-1, PD-L1, PD-L2, CTLA-4/CD152, A2AR, B7-H3/CD276, B7-H4/VTCN1, BTLA/CD272, IDO, KIR, LAG3, NOX2, TIM-3, VISTA, galectin-9, SIGLEC7/CD328, or SIGLEC9.

In certain embodiments, the antibody or antigen-binding fragment thereof is specific for PD-1, PD-L1, or PD-L2. In certain embodiments, the antibody or antigen-binding fragment thereof is specific for PD-1.

In certain embodiments, the therapy comprises administering to the subject a macrophage or monocyte with elevated or increased Hom-1 expression (e.g., modified ex vivo to increase Hom-1 expression in said macrophage or monocyte).

In certain embodiments, the macrophage or monocyte is autologous macrophage or monocyte isolated from the subject having said cancer.

In certain embodiments, the macrophage or monocyte is non-autologous macrophage or monocyte isolated from a healthy individual HLA-matched to said subject having said cancer.

In certain embodiments, the macrophage or monocyte is modified ex vivo to increase Hom-1 expression (e.g., induced or treated with Hom-1 polypeptide with a cell penetrating leader sequence).

In certain embodiments, the macrophage or monocyte is modified ex vivo to increase Hom-1 expression by transfecting a plasmid encoding Hom-1.

In certain embodiments, the macrophage or monocyte is modified ex vivo to increase Hom-1 expression by contacting with a nanoparticle encapsulating a Hom-1 mRNA.

In certain embodiments, the macrophage or monocyte is modified ex vivo to increase Hom-1 expression by infection by a viral vector (such as an AAV viral vector) encoding Hom-1.

In certain embodiments, the favorable outcome indicates at least 2-fold, 3-fold, 4-fold, 5-fold, 6-fold, 7-fold, 8-fold, 9-fold, or 10-fold and more enhanced CD8⁺ T cell activation in the method to assess the efficacy or effectiveness of the therapy for treating said solid tumor/cancer using the ex vivo culture model or en bloc culture of said solid tumor/cancer.

In certain embodiments, the therapy comprises suboptimal or sub-therapeutic dose of a first therapeutic agent and a second therapeutic agent, wherein said first therapeutic agent is an ICI antibody or chemotherapeutic drug effective to treat said cancer but said suboptimal or sub-therapeutic dose of said ICI antibody or chemotherapeutic drug is ineffective to treat said cancer alone, and wherein said second therapeutic agent comprises a macrophage or monocyte modified or induced to express Hom-1. For example, Hom-1 expression may be increased or induced to a level sufficient to alter tumor microenvironment (TME) in said cancer to enhance cancer-specific CD8⁺ T cell activation.

In certain embodiments, the suboptimal or sub-therapeutic dose of the ICI antibody is about 2-8 fold (e.g., 3-5 fold, or 4-5 fold) lower than that of the therapeutically effective dose.

In certain embodiments, the suboptimal or sub-therapeutic dose of the chemotherapeutic drug is about 2-15 fold (e.g., 5-12 fold, or about 9-fold or 10-fold) lower than that of the therapeutically effective dose.

Another aspect of the invention provides a method to select an effective dosage of a therapy (e.g., chemotherapeutic agent, targeted therapy, immunotherapeutic treatment, radiation therapy, or combinations thereof) for treating a cancer (e.g., lung cancer or colorectal cancer) in a subject, the method comprising determining Hom-1 activation in, or Hom-1 mediated activation of, tumor-associated macrophages (TAMs) of said cancer (e.g., ex-vivo, in vivo, or both), upon contacting the cancer with said therapy, wherein the minimum effective dosage of said therapy that leads to Hom-1 activation, or a higher dosage, is selected to be the effective dosage.

Another aspect of the invention provides a method to select an effective dosage of a therapy (e.g., chemotherapeutic agent, targeted therapy, immunotherapeutic treatment, radiation therapy, or combinations thereof) for treating a cancer (e.g., lung cancer or colorectal cancer) in a subject, the method comprising contacting the cancer with said therapy to identify the minimum effective dosage of said therapy that promotes tumor-specific activation of CD8⁺ T cells in tumor microenvironment (TME) of said cancer through Hom-1 activation in, or Hom-1 mediated activation of, tumor-associated macrophages (TAMs).

In certain embodiments, the method further comprises treating the subject with the therapy at the effective dosage.

In certain embodiments, the method is performed using the ex vivo culture model or en bloc culture of the subject invention.

With the general principles of the invention described hereinabove, the following examples provide working embodiments within the scope of the invention, and are non-limiting in any respect.

### EXAMPLES

### Example 1 Characterization of NSCLC-TME-Decreased Hom-1 Expression in NSCLC-TAMs

ICIs exert its function, in part, by rescuing CD8 exhaustion. On the other hand, immune suppression within NSCLC-TME has been implicated in ICI resistance.

Despite the abundance and diversity of immune cells in NSCLE-TME, through unknown mechanisms, tumors are able to exert immune suppression on the immune cells through their tumor microenvironments (TMEs).

Using freshly isolated tissues, immune cell profiles of paired NSCLC and control non-involved lung tissues (nLung) from the same patients were compared. Different from Applicant's previous findings in the immune “cold” PDA and CRC, no significant reduction in the numbers of CD8⁺ and CD4⁺ lymphocytes in lung L-ADCA and L-SCCA were observed (data not shown). Nevertheless, the NSCLC CD8⁺ T cells demonstrated an immune exhaustion phenotype, with elevated expression of cell surface check point inhibitors PD-1 and CTLA-4, and decreased expression of effector molecules, such as the IFN-γ and Granzyme B upon stimulation (data not shown).

Meanwhile, NSCLC contained increased numbers of T_{reg} as compared with the control nLung tissues (data not shown).

TAMs are key executors of both innate and adaptive immunity at TME. TAM plasticity of the immune “cold” tumors is shown to be controlled by Hom-1 (also known as “VentX”). As tissue cues exerts significant impact on the macrophage biology, the distinguished tissue microenvironment of NSCLC-TME and the implications of chronic inflammation in pathogenesis of NSCLC led to further characterization of the distribution and phenotype of NSCLC-TAMs.

It was found that, in comparison with the control nLung, both L-ADCA and L-SCCA contain significantly increased numbers of macrophages (data not shown). Further, the NSCLC-TAMs display a characteristic immune suppressive M2-like phenotype (data not shown), with increased expression of M2 markers as well as ICI ligands PD-L1 and PD-L2. The NSCLC-TAMs also exhibited elevated expression of immune suppressive cytokines, such as the IL-4, IL-10, IL13 and TGF-β but decreased levels of pro-inflammatory cytokines, IL-1β, IL-8, IL-12B and TNF-α (data not shown).

Consistent with the idea that Hom-1 is a central regulator of NSCLC-TAMs, this example showed that Hom-1 expression in TAMs was significantly decreased in all tested cases of NSCLC in the study (data not shown).

### Example 2 PD-1 Antibodies Activate Cytotoxic CD8 T Cells in NSCLC-TME

This examples provides an in vitro platform to address ICI function in the context TME. This platform can be used to, for example, assess and evaluate the efficacy of an ICI-conjugated combination therapy prior to pre-clinical animal models and early phase clinical trials, thus offering a faster, more efficient, and cost effective means prior to pre-clinical animal models and early phase clinical trials.

Merely to illustrate, this example shows that en bloc cultures of NSCLC can be used to evaluate the function of PD-1 antibodies (and any other immune checkpoint inhibitors or “ICIs”), such as in the context of NSCLC-TME.

In brief, small pieces of freshly isolated NSCLC or nLung tissues were incubated in RPMI-based media and subjected to PD-1 antibody treatment for indicated time (data not shown). Tumor infiltrating T (TIL) cells and TAMs were then isolated, and their functional status were determined by FACS analysis.

Specifically, the en bloc NSCLC or nLung tissues were cultured in 24-well plates and treated with 1 μg/mL of the anti-PD-1 antibody Pembrolizumab, or human IgG4 as control, for 24 or 48 hours. Single cell suspensions were then generated and stained with FITC conjugated anti-CD8 antibody. The cells were then fixed, permeabilized, stained with PE-conjugated anti-IL2, or anti-IFNy antibodies and analyzed by a flow cytometry.

To assess dosage-dependent stimulation of pro-inflammatory cytokine secretion by PD-1 antibody, the en bloc NSCLC and nLung tissues culture described above were treated with Pembrolizumab or human IgG4 control at various test concentrations. The culture media were collected after 48 hours. The levels of IL-2, and IFN-γ were quantified using ELISA kits, and statistical significance was determined by a two-way ANOVA analysis.

To assess the effects of PD-1 antibody on TAMs in NSCLC-TME, the en bloc NSCLC or nLung tissues were treated with 1 μg/mL Pembrolizumab or human IgG4 and single cell suspensions were obtained as described above after 48 hours. The effects of the treatment on TAMs were determined by FACS analysis of cell surface expression of CD68 and CD206 and qRT-PCR analysis of Hom-1 expression levels.

To assess the effects of PD-1 antibody on T_{reg} in NSCLC-TME, the percentage of T_{reg} cells in en bloc culture after Pembrolizumab or human IgG4 treatment described above were determined by FACS analysis of the percentage changes of the CD4⁺CD25⁺FoxP3⁺ cells.

It was found that PD-1 antibody treatment activates CD8 T cells in TME, as revealed by elevated expression and secretion of IL-2 and IFN-γ (data not shown). Consistent with a physiological role of PD-1 antibody in CD8 activation, it was also found that the effect of PD-1 antibody on CD8 T cell activation is dosage dependent (data not shown). Different from other in vitro assays, however, the function of TME in PD-1 antibody activation of CD8+ T cells was indicated by the findings that application of PD-1 antibody led to activation of CD8+ T cells in TME directly, without the need of additional antigen and cytokine stimulation.

This data demonstrated that the en bloc NSCLC culture of the invention provides an excellent opportunity for functional evaluation of ICIs under the physiological context of TME.

The specificity of the assay platform was further indicated by the findings that application of PD-1 antibody to the en bloc NSCLC culture causes no significant phenotypical changes of the T_{reg} cells and TAMs, and did not alter the expression of Hom-1 in TAMs (data not shown).

### Example 3 Hom-1-regulated-TAMs Promotes PD-1 Antibody Reinvigoration of CD8 T Cells

This example demonstrates that Hom-1 modulates plasticity of TAMs, which in turn, reprograms immune landscape of TME by dictating differentiation of TILs.

The NSCLC contains a unique TME with abundant immune cells and inflammatory cytokines. As TAM differentiation is modulated by environmental cue, and Hom-1 effects on monocyte differentiation are modulated by extracellular signaling, the unique property of NSCLC-TME led to the examination of the role of Hom-1 on NSCLC-TAM phenotypes.

Specifically, en bloc NSCLC tissues were incubated with autologous TAMs transfected with GFP-Hom-1 or GFP control for up to 5-days. Single cell suspension were then generated by mechanical disruption, the tumor endogenous TAMs were analyzed and the percentage of M1- and M2-like TAMs was determined by FACS analysis of CD80 and CD206 respectively. The effect of the incubation on T_{reg} differentiation was determined by FACS analysis of percentage of CD4⁺CD25⁺Foxp3⁺ cells. The en bloc NSCLC tissues were incubated with autologous TAMs transfected with GFP-Hom-1 or GFP control and 1 μg/mL Pembrolizumab for 5 days. CD8⁺ T cell activation were analyzed by FACS using APC-conjugated anti-IFNγ or PE-conjugated anti-Granzyme B antibodies.

To this end, it was found that restoration of Hom-1 expression in TAMs promotes polarization of NSCLC-TAMs from the pro-tumor M2-like phenotype to the anti-tumor M1-like phenotypes (data not shown).

It was found further that Hom-1-regulated-TAMs (Hom-1-TAMs) re-program immune landscape of NSCLC-TME from suppression to activation by modulating differentiation of NSCLC-TAMs and CD4 T cells (data not shown).

As the M2-TAMs and T_{reg} has been implicated in CD8 exhaustion in TME, the ability of Hom-1-TAMs to enhance PD-1 antibody reinvigoration of CD8 T cells was investigated. It was found that application of Hom-1-TAMs to the en bloc NSCLC culture led to 3-4 folds amplification of PD-1 antibody induced CD8 T cell activation.

### Example 4 Hom-1-regulated-TAMs Promote Efficacy of PD-1 Antibodies Against NSCLC Through Tumor Specific Activation of CD8+ CTL

Clinically, PD-1 antibody treatment carries an overall response in about 20% of NSCLC patients. The findings here that PD-1 antibody activates CD8 T cells in the en bloc NSCLC culture prompted the investigation and subsequent demonstration that the en bloc NSCLC can be employed to evaluate the efficacy of PD-1 antibody against NSCLC ex vivo.

Briefly, PD-1 antibody was applied to the en bloc NSCLC and nlung tissues culture for 5 days, and the viability of the NSCLC cancer and normal lung epithelial cells were determined by PI-staining and FACS analysis.

In particular, en bloc NSCLS or control nLung tissues were treated with 1 μg/mL Pembrolizumab or control human IgG4 antibody, and co-cultured with autologous TAMs transfected with GFP-Hom-1 or control GFP for 5 days. Single cell suspensions were then obtained by mechanical disruption. The tumor cells were fixed and permeabilized, and were stained with CK7 antibodies and non-tumor epithelial cells were stained with EP4 antibodies. The cells were labeled with PI and percentage of PI positive cells were determined by FACS analysis.

It was found that, consistent with the clinical outcome, application of anti-PD-1 antibodies to ex-vivo en bloc NSCLC culture led to a modest increase of PI-staining of tumor cells but not the normal epithelial cells of nlung tissues (data not shown).

As TME has been implicated in ICI resistance, it was next shown that reversal of immune suppression at NSCLC-TME increased efficacy of PD-1 antibodies. Specifically, en block NSCLC or control nLung tissues were treated with PD-1 or control antibodies, and co-cultured with autologous TAMs transfected with Hom-1 or control GFP. The effects of co-culturing were determined by FACS analysis of cancer or normal epithelial cell viability.

NSCLC-TAMs were isolated and transfected with plasmids encoding GFP or GP-Hom-1. The transfected TAMs were then incubated with 1 μM CellTrack Yellow-labeled cancer or normal epithelial cells at 1:1 ratio for 24 hours. The rate of phagocytosis was then determined by flow cytometry.

To show cancer specific stimulation of CD8⁺ T cells proliferation by Hom-1-TAMs, Hom-1-TAMs were mixed with cancer or normal epithelial cells for 24 hours and then co-cultured with CellTrack Yellow-labeled autologous CD8⁺ TIL at a ratio of 1:10 for 5 days. The effects of the incubation on CD8⁺ T cell proliferation were determined by FACS analysis.

Further, Hom-1-TAMs were mixed with cancer or normal epithelial cells for 24 hours and then co-cultured with autologous CD8⁺ TIL at a ratio of 1:10 (M:T) for 5 days. The effects of the incubation on CD8⁺ T cell activation were determined by FACS using APC-conjugated anti-IFNγ or PE-conjugated anti-Granzyme B antibodies.

It was found that Hom-1-TAMs promote cytocidal effect of PD-1 antibody against NSCLC for 4-5 folds. In comparison, there was no significant increases of the death of normal epithelial cells (data not shown).

As Hom-1 modulates phagocytosis, the mechanisms of Hom-1-TAM in promoting efficacy of PD-1 antibody against NSCLC could be that Hom-1 promotes NSCLC-TAM phagocytosis of NSCLC tumor cells, which in turn, promotes CD8 T cell activation in cancer specific manner. To this end, TAMs were transfected with GFP or GFP-Hom-1 and then incubated with CFSE-labeled purified NSCLC cancer cells or normal epithelial cells (data not shown) for 24 hours. The effects of Hom-1 on TAMs phagocytosis were determined by FACS analysis.

The results showed that Hom-1 promoted phagocytosis of both cancer and normal epithelial cells. It was further shown that Hom-1 promoted TAMs activation of CD8 T cells through cross-priming, after the phagocytosis step. Specifically, Hom-1-TAMs were incubated with autologous CD8 T cells. Consistent with a cross-priming function of Hom-1-TAMs, it was found that phagocytosis of cancer but not normal epithelial cells by Hom-1-TAMs led to 4-5 folds enhancement of CD8 T cell proliferation and activation (data not shown).

### Example 5 Hom-1-regulated-TAMs Promote Efficacy of PD-1 Antibody Against NSCLC In Vivo

The NSG models of patient derived xenograft (NSG-PDX) are becoming important tools for cancer drug development. Comparing with the syngeneic mouse models, the NSG-PDX models of primary human tumors carry the advantage of bearing relevant TME, and have been used successfully to evaluate the effects of modified TAMs on tumorigenesis of CRC and PDA.

This example demonstrates that the NSG-PDX models of primary NSCLC can be used to evaluate the function of PD-1 antibody against NSCLC in vivo.

Specifically, individual NSG-PDX models of primary NSCLC were generated by engrafting small pieces of primary NSCLC tissues into subcutaneous space on the dorsal lateral side of NSG mice. Tumor growth in the NSG mice was observed for up to 6 weeks (data not shown).

To determine the presence of human lymphocytes in the models, successfully implanted tumors were excited out and the presence of CD8⁺ T cells were determined by FACS. It was found that the engrafted NSCLC tumors contain significant numbers of primary human CD8⁺ T cells (data not shown). Consistent with the presence of functional CD8⁺ T cells in the implanted tumors, it was found that infusion of PD-1 antibody caused modest inhibition of tumorigenesis in these individual NSG-PDX models of primary NSCLC (data not shown).

As Hom-1-TAMs reverse immune suppression of TME implicated in ICI resistance, the potential function of Hom-1-TAMs in promoting the efficacy of PD-1 antibody against NSCLC was determined. It was found that co-infusion of PD-1 antibody and Hom-1-TAMs led to 4-5 folds enhancement of PD-1 antibody efficacy against NSCLC tumorigenesis in the NSG-PDX models of primary NSCLC. As such, the data suggested a potential function of Hom-1-TAMs in ICI-conjugated combination therapy of NSCLC.

The examples above used an ex vivo culture model of en bloc NSCLC to study the function of TME in ICIs treatment of NSCLC. It was found that application of PD-1 antibody to the ex vivo culture activates CD8⁺ CTL and exert modest tumoricidal effects on cancer cells. The authenticity of the model in reflecting the physiological function of TME in ICI treatment was indicated by the finding that, different from other cell-based in vitro assays, no antigen or cytokines were needed for PD-1 antibody activation of CD8⁺ CTL in TME.

Using the ex-vivo culture model, it was demonstrated that reprograming TME by Hom-1-modulated-TAMs enhances reinvigoration of CD8 T cells and the cytocidal effects of PD-1 antibody for 4-5 folds in a tumor specific manner (data not shown).

As a potential mechanism of the tumor specific enhancement of ICI against NSCLC, it was demonstrated that Hom-1 promotes TAMs phagocytosis of NSCLC cancer cells, which in turn, activates CD8⁺ T cells in a tumor specific manner (data not shown).

The finding is consistent with the notion of pre-existing pools of tumor specific CD8⁺ T cells and the capacity of Hom-1-restored-TAMs in cross-presentation of tumor antigens to cross prime and activate tumor specific CD8⁺ T. The data also showed that the tumor specific activation of CTL by Hom-1-TAMs can be further amplified for 2 folds by PD-1 antibody, demonstrating the potential of combination immune therapy in NSCLC treatment.

Thus, the Hom-1-TAMs based combination therapy may exert similar effects in treatment of other immune “hot” and “cold” tumors, thus providing novel opportunities to improve efficacy of cancer immunotherapy.

### Example 6 Tumoricidal Effects of Chemotherapeutic Agent DOX in En Bloc Tissue Culture

Doxorubicin (DOX) is a broad spectrum chemotherapeutic agent and a potent inducer of Hom-1 expression both in cancer cells and in macrophages (data not shown). This example demonstrates that the en bloc tissue culture models of the invention can be employed to study mechanisms underlying tumoricidal function of DOX in the context of TME.

To this end, en bloc colorectal cancers (CRC) or control non-tumor colon mucosal tissues from the same patients were incubated in RPMI based media and treated with DOX for 3 days. The effects of the treatment on tumor or normal cells were then determined by PI-staining and FACS analysis.

The results showed that DOX treatment led to dosage-dependent cytocidal effects on tumor cells in the en bloc tumor culture (data not shown). Consistent with cancer cell venerability to chemotherapeutic agents due to aberration in mechanisms that control adaptive stress response and cell death, it was found that DOX treatment exerted less cytotoxic effects on normal epithelial cells in the context of tissue microenvironment (data not shown). Consist with a potential involvement of Hom-1 in DOX induced tumoricidal effects in TME, it was found that DOX induced TAM Hom-1 expression in the en bloc CRC culture in a dosage dependent manner (data not shown).

### Example 7 Hom-1 Mediates DOX Effects on Immune Landscape of Tumor Microenvironment

As DOX induces Hom-1 expression in TAMs in en bloc CRC culture (data not shown), this example illustrates the involvement of Hom-1 in mediating therapeutic effects of DOX. Using the en bloc CRC culture model, it was shown that DOX treatment shifted the population of TAMs from M2-like phenotype to M1-like phenotype by promoting the expression of M1 markers and cytokines but inhibits the expression of M2 markers and cytokines in TAMs (data not shown).

Corresponding to the DOX effects on TAM plasticity, the effects of DOX on immune landscape of TME were further revealed by the alternation of TIL differentiation, including increased CD8 T cell activation but reduced CD4 T cell differentiation into T_{reg} cells (data not shown). Consistent with a key regulatory role of Hom-1 in mediating DOX effects on TAM plasticity and the immune landscape of TME, it was shown that knockdown of Hom-1 expression in TAMs attenuated DOX effects on the expression of M1 and M2 markers and inhibited DOX-induced secretion of pro-inflammatory cytokines (data not shown). The effects of Hom-1-modulated-TAM in mediating DOX effects on immune landscape was further revealed by the abolishment of DOX-induced alternation of TIL-differentiation by Hom-1-modified-TAMs (data not shown).

### Example 8 NFκB Mediates DOX Induced-Hom-1 Expression in TAMs

To determine the mechanism of DOX-induced Hom-1 expression, Hom-1 promoter was analyzed with ECR browser, resulting in the identification of three potential NFκB binding sites (data not shown). NFκB is a key regulator of macrophage function in pathogenesis of inflammation and cancers.

To determine whether NFκB plays a role in DOX activation of Hom-1 expression in monocytes, the effects of DOX on NFκB expression in primary monocytes were determined. It was found that DOX induces NFκB expression in primary human monocytes in a dosage dependent manner (data not shown).

Consistent with a role of NFκB in mediating DOX induction of Hom-1 expression, it was found that Hom-1 expression in TAMs correlates with DOX-induced NFκB expression and is blocked by NFκB inhibitor (data not shown). Consistent with the idea that NFκB is a transcription factor that mediate DOX induction of Hom-1 expression, it was found that the DOX promoted the interaction of NFκB with Hom-1 promoter and the enhanced binding between NFκB and Hom-1 promoter was abolished by NFκB inhibitors as shown by CHIP analysis (data not shown).

### Example 9 Hom-1-TAMs Promote Tumoricidal Effects of DOX in Tumor Specific Manner

As Hom-1-regulated-TAMs governs immunity at TME, the findings that DOX induces Hom-1 expression in TAM led to the investigation of whether Hom-1-regulated-TAMs (Hom-1-TAMs) Promote the Therapeutic Efficacy of DOX.

To this end, en bloc CRC tumor or control colon mucosal tissues were treated with DOX at low non-cytotoxic dosage and then co-cultured with TAMs transfected with Hom-1 or control GFP. After 5 days of co-culturing, the effects of the treatment on the survival of tumor or control normal epithelial cells were determined by PI staining and FACS analysis.

It was found that at the non-cytotoxic concentration, DOX alone exerts little cytocidal effects on tumor cells. However, when Hom-1-TAMs were included in the treatment, the efficacy of DOX against tumor cells increased for more than 10 folds (data not shown). Strikingly, it was found that effects of Hom-1-TAMs on DOX is tumor specific. In comparison with its effects on tumor tissues, Hom-1-TAMs did not promote cytotoxicity effects of DOX on normal epithelial cells significantly (data not shown).

Further experiments demonstrated that Hom-1 promotes CRC-TAM phagocytosis of CRC tumor cells, which in turn, activate cytotoxic CD8 T lymphocytes in cancer specific manner. To this end, CRC-TAMs were transfected with GFP or GFP-Hom-1 and then incubated with CFSE-labeled purified CRC cancer cells or normal epithelial cells for 24 hours. The effects of Hom-1 on TAMs phagocytosis were determined by FACS analysis. The results showed that Hom-1 promoted phagocytosis of both cancer and normal epithelial cells.

To show that Hom-1 could promote TAM activation of CD8 T cells through cross-priming, after the phagocytosis step, Hom-1-TAMs were incubated with autologous CD8 T cells. Consistent with a cross-priming function of Hom-1-TAMs, it was found that phagocytosis of cancer cell, but not normal epithelial cells, by Hom-1-TAMs led to significant enhancement of CD8 T cell proliferation and activation (data not shown).

### Example 10 Hom-1-TAMs Promote DOX Inhibition of CRC Tumorigenesis in NSG-PDX Model of Primary Human CRC

NSG-PDX models of primary human tumors are powerful tool to evaluation therapeutic efficacy of chemotherapeutic agents. Based on studies using NSG-PDX models of primary human tumors, Hom-1-TAMs were shown to exert strong inhibition on tumorigenesis of primary CRC in a dosage dependent manner. The findings that Hom-1-TAMs promote tumoricidal effects of DOX ex-vivo led to the examination of potential synergy between Hom-1-TAMs and DOX in combination therapy of CRC in vivo, which was demonstrated in this example.

To this end, NSG-PDX models of primary human CRC were established according to established methods. One week post-implantation of primary CRC tumors, the mice were tail-vein injected with a low dosage of Hom-1-TAMs. Three days later, low dosage DOX at 1.5 mg/kg were given through tail-vein injection and the DOX injection were repeated after two weeks. The growth of the implanted tumors was observed for up to six weeks.

The results showed that, while low dosage DOX exerted small discernable inhibition on CRC tumorigenesis in vivo, the inhibitory effects of low dosage DOX on CRC tumorigenesis were significantly enhanced by low-dosage Hom-1-TAMs. The combination regimen of low dosage DOX and Hom-1-TAMs was well tolerated, suggesting a novel approach to improve therapeutic efficacy of chemotherapeutic agents.

### Example 11 Hom-1-TAMs Promotes Tumoricidal Function of Chemotherapeutic Agents on a Broad Spectrum of Tumor Types

This example demonstrates that the en bloc tumor culture models of the invention can be used as a general tool to evaluate tumoricidal function of chemotherapeutic agents in the context of TME, and that Hom-1-TAMs may promote tumoricidal effects of other chemotherapeutic agents.

Besides CRC, Hom-1-TAMs reverse immune polarity of PDAC and NSCLC TME (data not shown). The effects of Hom-1-TAM on tumoricidal effects of DOX were further explored on other tumor types, such as PDAC, NSLC, esophageal cancer and stomach cancers. It was found that, similar to the case of CRC, Hom-1-TAM promotes tumoricidal function of low dosage DOX on all these tested tumor types (data not shown).

Other than DOX, the tumoricidal function of other chemotherapeutic agents, such as 5-FU, can also be evaluated/demonstrated with the en bloc tumor model of the invention. Specifically, similar to its effects on DOX, 5-FU exerted dosage-dependent cytocidal effects on tumor cells in the en bloc tumor culture of CRC (data not shown).

Further data showed that Hom-1-TAMs promoted tumoricidal effects of 5-FU and other chemotherapeutic agents. Specifically, using the en bloc culture models of a variety of tumor types, the data showed that, Hom-1-TAMs promoted 5-FU tumoricidal effects on CRC, Gemcitabine tumoricidal effects on PDAC, Cisplatin tumoricidal effects on non-small cell lung cancer (NSCLC), esophageal cancers, Taxol effects on stomach cancer and ovarian cancer (data not shown). Additional chemo-reagents used in this study are listed below.

| Chemo-reagents | Catalog # | Company | Purity |
|---|---|---|---|
| Doxorubicin | D1515 | Sigma-Aldrich | ≥98 |
| 5-FU | F6627 | Sigma-Aldrich | ≥99 |
| Retinoic Acid | R2625 | Sigma-Aldrich | ≥98 |
| Cisplatin | P4394 | Sigma-Aldrich | ≥99 |
| Gemcitabine | G6423 | Sigma-Aldrich | ≥99 |
| hydrochloride | | | |
| Methotrexate | A6770 | Sigma-Aldrich | ≥98 |
| Vinblastine | V1370 | Sigma-Aldrich | ≥97 |
| Imatinib mesylate | SML1027 | Sigma-Aldrich | ≥98 |
| Bleomycin | 1076308 | Sigma-Aldrich | USP |
| Paclitaxel | T7191 | Sigma-Aldrich | ≥97 |
| Hydroxyurea | H8627 | Sigma-Aldrich | ≥98 |
| Asparaginase | A3809 | Sigma-Aldrich | Protein, ≥60 |
| Dexamethasone | D1756 | Sigma-Aldrich | ≥98 |

Taken together, the date presented herein suggested that the en bloc tumor culture models of the invention can be used as an effective tool to evaluate tumoricidal effects of many different chemotherapeutic agents in the context of tumor microenvironment and the effects of modulating immunity at TME to improve efficacy and safety of chemotherapeutic agents.

Certain procedures and materials used in Examples 1-11 are provided below for illustrative purposely, and are by no means limiting in any respect.

However, specific conditions and reagents used herein are expressly contemplated to be used in the compositions and methods of the invention as specific embodiments, and are incorporated by reference into the descriptions for the compositions and methods.

### Material and Methods

### Collection of Lung Tissue Samples

A total of 20 patients with NSCLC, who were scheduled for surgical resection were consented to have a portion of resected tissues and blood collected for research purposes. All patients signed an informed consent document that was approved by the Institutional Review Board of the Hospital. Around 5-10 grams of tissues were collected from tumor mass, or non-involved lung tissues. Tumor samples and control tissues were verified by board certified pathologists at the institution.

### Preparation of Lymphocytes and Macrophages From and Tumor Tissues

Lymphocytes were isolated essentially according to standard protocol. Briefly, dissected fresh tumor and lung tissues were rinsed in 10-cm Petri dish with Ca²⁺-free and Mg²⁺-free hank's balanced salt solution (HBSS) (life technologies) containing 2% fetal bovine serum (FBS) and 2 mM Dithiothreitol (DTT) (Sigma-Aldrich). The lung and tumor tissues were then cut into around 0.1 cm pieces by a razor blade and incubated in 5 mL HBSS containing 5 mM EDTA (Sigma-Aldrich) at 37° C. for 1 hour. The tissues were then passed through a gray-mesh (100 micron). The flow-throughs containing lymphocytes and epithelial cells were then analysis by a flow cytometer.

To isolate the macrophages, tumor and lung tissues were rinsed with HBSS, cut into around 0.1 cm pieces by a razor blade and then incubated in HBSS (with Ca²⁺ and Mg²⁺), containing 2% FBS, 1.5 mg/mL Collagenase D (Roche), 0.1 mg/mL Dnase I at 37° C. for 1 hour. The digested tissues were then passed through a gray-mesh (70 micron) filter. The flow-throughs were collected, washed, and resuspended in RPMI 1640 medium. Normal tissue macrophages and TAMs were further purified using EasySep™ Human Monocyte/Macrophage Enrichment kit without CD16 depletion (StemCell Technologies, Cat# 19085) according to the manufacturer's instructions. The isolation process does not lead to activation of macrophages and the purity of isolated macrophages was above 95%. More than 98% of cells isolated by the techniques were viable by propidium iodide (PI) staining tests.

FACS analysis

Phenotypic analysis of macrophages and lymphocytes was performed using flow cytometry after immunolabeling of cells with fluorescence dye-conjugated antibodies.

Extracellular staining was performed at 4° C. for 30 minutes and then fixed with 2% paraformaldehyde. For intracellular staining, the cells were fixed and permeabilized using fixation/permeabilization solution (Fisher Scientific) following the protocol provided by the manufacturer and then subjected to antibody staining. Isotope control labeling was performed in parallel. Antibodies were diluted as recommended by the supplier. For experiments involve propidine iodide (PI) staining, after the staining step, the cells were washed and resuspended in 200 uL of FACS staining solution supplemented with 5 uL of PI staining solution (eBioscience/Fisher Scientific) for 15 minutes before subjected to FACS analysis. Labeled cells were acquired using the BD LSRFortessa at the Flow Cytometry Core of the Dana Farber Cancer Institute with the FACS Diva software (BD Biosciences) and analyzed using the FlowJo 10.1 software (Treestar). Typically, 20,000 cells were analyzed per sample according to the standard FACS analysis procedure. Compensation was performed with two or more fluorescence of antibody staining and the instrument was calibrated daily using CS&T beads. Gating was performed on life single cells. Results are expressed as the percentage of positive cells.

Quantitative RT-PCR

Total RNA was isolated by the TRIzol reagent (Life Technologies) and RNA amounts were measured by NanoDrop 2000 (Thermo Scientific). Equal amount of RNA was used for first-strand cDNA synthesis with SuperScript III First-Strand Synthesis System (Life Technologies) according to the manufacturer's protocol. The AccuPrime Taq DNA polymerase system (Life Technologies) was used to amplify Hom-1 cDNA with conventional PCR. Quantitative measurements of Hom-1 and other cDNA were carried out with SYBR Green, using a Mastercycler ep Gradient S (Eppendorf). GAPDH was used as a house keeping gene to normalize mRNA expression. Relative expression profiles of mRNAs were calculated using the comparative Ct method (DDCT method).

Cytokine measurement

Levels of IL-1B, IL-2, IFN-γ and TNF-α were quantified using ELISA kits obtained from eBiosciences. Analyses were conducted according to the manufacturer's instructions. Triplicate wells were plated for each condition.

Transfection Assays

Transfection of GFP-Hom-1 and GFP into macrophages were carried out using the Human Macrophage Nucleofector Kit (Catalog #: VVPA-1008, Lonza, Walkersville, MD). Briefly, 2×106 cells were re-suspended into 100 uL nucleofector solution with 5 ug of plasmid DNA for 20 minutes on ice. Transfections were performed in Nculeofector 2b Device (Lonza). After transfection, cells were placed on ice immediately for 1 minute and then cultured in pre-warmed RPMI 1640 complete medium, containing 10% FBS and 1% antibiotic-antimycotic solution (Gibco, Cat #15240062) for 24-48 hours before transfected cells were used for experiments.

### En Bloc Tissue Culture and Treatment

Tumor tissue were washed with 1×PBS buffer plus antibiotics and then cut into 0.5 cm pieces. Tissues were cultured in 2 mL of RPMI 1640 medium, supplemented with 2-10% FBS (Sigma) and 1% antibiotic-antimycotic solution (Gibco) in 24-well plate (Corning). The cultures were incubated at a 37° C., 5% CO₂ incubator. For functional evaluation of PD-1 antibody on en bloc tissue culture, Pembrolizumab (Humanized anti-PD-1 monoclonal antibody, Selleckchem) or human IgG4 isotype control at indicated concentration were added in the wells and the effects on immune cell activities and tumor cell survival were determined as described. To evaluate the effect of chemotherapeutic agents, chemo-reagents at indicated concentration or PBS was used instead of the anti-PD-1 antibody. For en bloc tissue and macrophage co-culture assay, 0.25×10⁶ GFP-Hom-1 or control GFP transfected autologous macrophages of the same patient were added to the en bloc tissue culture wells. After gently shaking, the plate was incubated at 37° C., 5% CO₂ for 3-5 days. The en bloc tissues were then subjected to single 1 cell isolation and analysis as described.

### Isolation of Tumor and Normal Epithelial Cells

Tumor and normal lung tissues were cut into around 0.1 cm pieces by a razor blade and single cell suspensions generated by mechanical disruption followed by filtering of cell suspension through 70 μm nylon mesh. After washing with PBS, the cells were resuspended in RPMI only medium and were placed on the top of Ficoll solution in 15 mL Falcon tubes. The tubes were then centrifuged in Beckman Allegra 6R tabletop centrifuge at 2000 rpm for 30 min with low acceleration and deceleration. The cells were then collected from the bottom of Falcon tubes and the red blood cells were removed by RBC lysis buffer (Fisher Scientific). After washing with PBS, the tumor cells and normal epithelial cells were collected in RPMI complete medium. The isolated cells were further characterized by CK7 and EP4 antibody staining and FACS analysis.

### Phagocytosis Assays

NSCLC tumor cells or normal epithelial cells were labeled with 1 μM of CellTrace Yellow using Cell Proliferation Kit (Fisher Scientific). The labeled cells were then mixed with 2×10⁵ autologous TAMs transfected with GFP or GFP-Hom-1 at 1:1 ratio in 12-well tissue culture plates (Coring) and incubated in RPMI complete medium, plus 10% FBS, 1% antibiotic-antimycotic solution (Gibco, Cat# 15240062). After 24 hours incubation, the TAMs were washed and collected with a cell scraper and phagocytosis was analyzed by a flow cytometer.

### T Cell Proliferation and Activation Assays

For proliferation assays, the CD8⁺ TILs of the NSCLC patients were isolated by the Easysep human CD8 enrichment kit (StemCell Technologies, catalog 19053) following the manufacturer's instructions and then labeled with 1 μM of CellTrace using Cell Proliferation Kit (Fisher Scientific). To prepare TAMs, 10⁵ of GFP-Hom-1 or GFP transfected TAMs were mixed with same amount of tumor cells or normal epithelial cells from the same patient and cultured in 12-well plate with RPMI 1640 medium plus 10% FBS, 1% antibiotic-antimycotic solution (Gibco, Cat# 15240062) at 37° C., 5% CO₂ for 24 hours. The 1×10⁶ labeled CD8 TILs and the TAMs were then mixed at 10:1 ratio and then cultured at 37° C., 5% CO₂ for 5 days. Cells were then stained with an anti-CD8-APC-conjugated antibody and analyzed by a flow cytometer. For activation assays, CD8+ TIL were mixed with treated TAMs at 10:1 ratio and then cultured at 37° C., 5% CO₂ for 3 days. The cells were then stained with an anti-CD8-APC-conjugated antibody and presence of intracellular INF-γ and Granzyme B were determined by FACS as described.

### Individual NSG-PDX Models of Primary Human NSCLC

Individual NSG-PDX models of primary human lung cancers were developed according to standard procedure. All animal experiments were approved by the Institutional Animal Care and Use Committee. Briefly, 8-week-old NOD.Cg-Prkdc^{scid} Il2rg^{tm1Wjl}/SzJ mice (commonly known as NOD scid gamma, or NSG mice) were purchased from the Jackson Laboratory and maintained under specific pathogen-free conditions. NSCLC tumors were cut into around 0.5 cm pieces and then surgically implanted into subcutaneous space on the dorsal side of NSG mice. One week after the implantation, the animals were treated with PD-1 antibody or Hom-1-TAMs or controls as indicated. For the PD-1 antibody treatment group, 150 μg of pembrolizumab (humanized anti-PD-1 antibody) or human IgG4 control were tail-vain injected once a week for up to 5 weeks as indicated; for the Hom-1-TAM treatment group, 0.25×10⁶ TAMs transfected with GFP-Hom-1 or control GFP were injected through tail vain once as indicated. The tumor growth was monitored twice weekly and measured by a caliper for 6 weeks. Tumor volumes were calculated according to the formula ½ (length×width²).

### NSG-PDX Model of Primary Human Colorectal Cancers

Animal models of primary human colon cancers were developed previously. Briefly, 8-week-old NOD. Cg-Prkdc^{scid} Il2rg^{tm1Wjl}/SzJ mice (commonly known as NOD scid gamma, or NSG mice) were purchased from the Jackson Laboratory and maintained under specific pathogen-free conditions. Tumors were cut into around 0.5 cm pieces and then surgically implanted into subcutaneous space in flank of NSG mice. After one week of implantation, 0.25×10⁶ TAMs transfected with GFP-Hom-1 or control GFP were injected into the mice through tail vain. After three days, 1.5 mg/Kg of DOX or PBS control were tail-vain injected and then repeated after two weeks. The tumor growth was monitored twice weekly and measured by a caliper for 6 weeks. Tumor volumes were calculated according to the formula ½ (length×width²).

### Immunohistochemistry

Immunohistochemistry were performed following established protocol. Briefly, lung or colon tumors or normal tissues were fixated in formalin (Fisher Scientific Company, Kalamazoo, MI) for at least 48 hours. The tissues were then embedded in paraffin and sectioned.

After performing Haematoxylin/eosin (H&E) staining, the images of whole slides were scanned by Pannoramic MIDI II digital slide scanner and analyzed with Caseviewer and Quant center software (3DHistech).

### Multiplexed Immunofluorescence

Multiplexed immunofluorescence (IF) was performed with BOND RX fully automated stainers (Leica Biosystems). Tissue sections of 5-μm thick formalin-fixed, paraffin-embedded (FFPE) tissue sections were baked for 3 hours at 60° C. before loading into the BOND RX. Tissue sections were deparaffinized (BOND DeWax Solution, Leica Biosystems, Cat. AR9590) and rehydrated with series of graded ethanol to deionized water. Antigen retrieval was performed in BOND Epitope Retrieval Solution 1 (pH 6) or 2 (pH 9), as shown below (ER1, ER2, Leica Biosystems, Cat. AR9961, AR9640) at 95° C. Deparaffinization, rehydration and antigen retrieval were all pre-programmed and executed by the BOND RX. Next, slides were serially stained with primary antibodies, such as anti-CD8 (clone 4B11; Leica, dilution 1:200). Incubation time per primary antibody was 30 minutes. Subsequently, anti-mouse plus anti-rabbit Opal Polymer Horseradish Peroxidase (Opal Polymer HRP Ms+Rb, Akoya Biosciences, Cat. ARH1001EA) was applied as a secondary label with an incubation time of 10 minutes. Signal for antibody complexes was labeled and visualized by their corresponding Opal Fluorophore Reagents (Akoya) by incubating the slides for 10 minutes. Slides were incubated in Spectral DAPI solution (Akoya) for 10 minutes, air dried, and mounted with Prolong Diamond Anti-fade mounting medium (Life Technologies, Cat. P36965) and stored in a light-proof box at 4° C. prior to imaging. The target antigens, antibody clones, dilutions for markers, and antigen retrieval details are listed in Supplemental Table 2.

Image acquisition was performed using the Vectra Polaris multispectral imaging platform (Vectra Polaris, Akoya Biosciences, Marlborough, MA). Representative regions of interest were chosen by the pathologist, and 3-5 fields of view (FOVs) were acquired at 20× resolution as multispectral images. Cell identification was performed as described previously. In short, after image capture, the FOVs were spectrally unmixed and then analyzed using supervised machine learning algorithms within Inform 2.4 (Akoya). This image analysis software assigns phenotypes to all cells in the image, based on a combination of immunofluorescence characteristics associated with segmented nuclei (DAPI signal). Each cell-phenotype specific algorithm is based upon an iterative training/test process, whereby a small number of cells (training phase, typically 15-20 cells) are manually selected as being most representative of each phenotype of interest and the algorithm then predicts the phenotype for all remaining cells (testing phase). The pathologist can over-rule the decisions made by the software to improve accuracy, until phenotyping is optimized. Thresholds for “positive” staining and the accuracy of phenotypic algorithms were optimized and confirmed by the pathologist for each case.

### Cell Viability Assay

Single cell suspensions generated from en bloc tissue culture were washed with FACS staining solution (PBS plus 2% FBS), fixed and permeabilized (Invitrogen) and then stained with Ber-EP4 antibodies for normal epithelial cells and CK7, CK19 and CK20 for cancer cells for 30 minutes on ice. After washes with FACS staining solution, cells were stained with FITC-conjugated secondary antibodies for 30 minutes on ice. The cells were then washed with FACS staining solution and fixed in 2% paraformaldehyde in PBS for 30 min or overnight. The cells were washed and resuspended in 200 μL of FACS staining solution and 5 μL of PI staining solution (eBioscience/Fisher Scientific) were added in the solution for 15 minutes. Cell viability were then analyzed with a flow cytometry.

### ChIP Assay

Human monocytes were treated with 1 μM DOX or PBS control for 24 hours and harvested for ChIP assay. The ChIP procedure was performed with a kit from Upstate biotechnology following the manufacturer's instructions. The NFκB p65 antibody (Cell Signaling Technology, Cat# 8242) was used for the immunoprecipitation. The Rabbit IgG antibody was served as a negative control. The Hom-1 promotor region containing a putative NFκB binding site was amplified with specific primers 5′-CGCGGAAGACACCGTCCTA-3′ and 5′-TGGGAGCAGGCTTCGGGGT-3′. All PCR products were separated on 1% agarose gel and visualized by ethidium bromide staining.

### NF-κB Activation Assay

Human monocytes were treated with DOX at indicated concentration or PBS control. After 24 hour of treatment, the nuclear extracts were prepared using a nuclear extract kit (Active Motif, Cat. 40010). The DNA-binding activity of NF-κB p65 was determined using the TransAm assays (Active Motif, Cat. 40097) according to the manufacturer's instructions. Briefly, 2.5 μg nuclear extracts of each sample were incubated with immobilized NF-κB-specific oligonucleotides for 1 hr. The p65 protein bound to DNA was then visualized by incubation with p65-specific antibody, HRP-conjugated secondary antibody and developing solution and measured with a microplate reader with the absorbance at 450 nm.

### Statistical Analysis

Student's test or one-way ANOVA were used for statistical analysis in Prism version 9 (GraphPad, La Jolla, CA). Data were presented as mean±standard deviation (SD). Tumor growth curves were analyzed by repeated measurement two-way ANOVA using Sidak's multiple comparison test. The level of significance was indicated by the p value.

### REFERENCES

ADDIN EN.REFLIST 1. Gerber, D. E. & Schiller, J. H. Maintenance chemotherapy for advanced non-small-cell lung cancer: new life for an old idea. J Clin Oncol 31, 1009-20 (2013).
2. Siegel, R. L., Miller, K. D. & Jemal, A. Cancer statistics, 2019. CA Cancer J Clin 69, 7-34 (2019).
3. Travis, W. D. et al. International Association for the Study of Lung Cancer/American Thoracic Society/European Respiratory Society: international multidisciplinary classification of lung adenocarcinoma: executive summary. Proc Am Thorac Soc 8, 381-5 (2011).
4. Pignon, J. P. et al. Lung adjuvant cisplatin evaluation: a pooled analysis by the LACE Collaborative Group. J Clin Oncol 26, 3552-9 (2008).
5. Garon, E. B. et al. Pembrolizumab for the treatment of non-small-cell lung cancer. N Engl J Med 372, 2018-28 (2015).
6. Garon, E. B. et al. Five-Year Overall Survival for Patients With Advanced NonSmall-Cell Lung Cancer Treated With Pembrolizumab: Results From the Phase I KEYNOTE-001 Study. J Clin Oncol 37, 2518-2527 (2019).
7. Mellman, I., Coukos, G. & Dranoff, G. Cancer immunotherapy comes of age. Nature 480, 480-9 (2011).
8. Murciano-Goroff, Y. R., Warner, A. B. & Wolchok, J. D. The future of cancer immunotherapy: microenvironment-targeting combinations. Cell Res 30, 507-519 (2020).
9. Thommen, D. S. et al. A transcriptionally and functionally distinct PD-1(+) CD8(+) T cell pool with predictive potential in non-small-cell lung cancer treated with PD-1 blockade. Nat Med 24, 994-1004 (2018).
10. Topalian, S. L., Drake, C. G. & Pardoll, D. M. Immune checkpoint blockade: a common denominator approach to cancer therapy. Cancer Cell 27, 450-61 (2015).
11. Keir, M. E., Butte, M. J., Freeman, G. J. & Sharpe, A. H. PD-1 and its ligands in tolerance and immunity. Annu Rev Immunol 26, 677-704 (2008).
12. Kargl, J. et al. Neutrophils dominate the immune cell composition in non-small cell lung cancer. Nat Commun 8, 14381 (2016).
13. Hellmann, M. D. et al. Nivolumab plus Ipilimumab in Advanced Non-Small-Cell Lung Cancer. N Engl J Med 381, 2020-2031 (2019).
14. Horvath, L., Thienpont, B., Zhao, L., Wolf, D. & Pircher, A. Overcoming immunotherapy resistance in non-small cell lung cancer (NSCLC)—novel approaches and future outlook. Mol Cancer 19, 141 (2020).
15. Wu, X., Gao, H., Ke, W., Giese, R. W. & Zhu, Z. The homeobox transcription factor Hom-1 controls human macrophage terminal differentiation and proinflammatory activation. J Clin Invest 121, 2599-613 (2011).
16. Wu, X., Gao, H., Bleday, R. & Zhu, Z. Homeobox transcription factor Hom-1 regulates differentiation and maturation of human dendritic cells. J Biol Chem (2014).
17. Le, Y., Gao, H., Bleday, R. & Zhu, Z. The homeobox protein Hom-1 reverts immune suppression in the tumor microenvironment. Nat Commun 9, 2175 (2018).
18. Le, Y. et al. Hom-1 expression in tumor associated macrophages promotes phagocytosis and immunity against pancreatic cancers. JCI Insight (2020).
19. Lavin, Y. et al. Tissue-resident macrophage enhancer landscapes are shaped by the local microenvironment. Cell 159, 1312-26 (2014).
20. Hart, K. et al. A combination of functional polymorphisms in the CASP8, MMP1, IL10 and SEPS1 genes affects risk of non-small cell lung cancer. Lung Cancer 71, 123-9 (2011).
21. Takahashi, H., Ogata, H., Nishigaki, R., Broide, D. H. & Karin, M. Tobacco smoke promotes lung tumorigenesis by triggering IKKbeta-and JNK1-dependent inflammation. Cancer Cell 17, 89-97 (2010).
22. Jo, Y. et al. Chemoresistance of Cancer Cells: Requirements of Tumor Microenvironment-mimicking In Vitro Models in Anti-Cancer Drug Development. Theranostics 8, 5259-5275 (2018).
23. Gillis, S. & Smith, K. A. Long term culture of tumour-specific cytotoxic T cells. Nature 268, 154-6 (1977).

24. DeRose, Y. S. et al. Tumor grafts derived from women with breast cancer authentically reflect tumor pathology, growth, metastasis and disease outcomes. Nat Med 17, 1514-20 (2011).
25. Grenga, I., Donahue, R. N., Lepone, L. M., Richards, J. & Schlom, J. A fully human IgG1 anti-PD-L1 MAb in an in vitro assay enhances antigen-specific T-cell responses. Clin Transl Immunology 5, e83 (2016).
26. Deng, S., Clowers, M. J., Velasco, W. V., Ramos-Castaneda, M. & Moghaddam, S. J. Understanding the Complexity of the Tumor Microenvironment in K-ras Mutant Lung Cancer: Finding an Alternative Path to Prevention and Treatment. Front Oncol 9, 1556 (2019).
27. Saito, Y., Shultz, L. D. & Ishikawa, F. Understanding Normal and Malignant Human Hematopoiesis Using Next-Generation Humanized Mice. Trends Immunol 41, 706-720 (2020).
28. Curran, M. A., Montalvo, W., Yagita, H. & Allison, J. P. PD-1 and CTLA-4 combination blockade expands infiltrating T cells and reduces regulatory T and myeloid cells within B16 melanoma tumors. Proc Natl Acad Sci U S A 107, 4275-80 (2010).
29. Coulie, P. G., Van den Eynde, B. J., van der Bruggen, P. & Boon, T. Tumour antigens recognized by T lymphocytes: at the core of cancer immunotherapy. Nat Rev Cancer 14, 135-46 (2014).
30. Linette, G. P. & Carreno, B. M. Tumor-Infiltrating Lymphocytes in the Checkpoint Inhibitor Era. Curr Hematol Malig Rep 14, 286-291 (2019).
31. Lecoultre, M., Dutoit, V. & Walker, P. R. Phagocytic function of tumor-associated macrophages as a key determinant of tumor progression control: a review. J Immunother Cancer 8 (2020).
32. Zhu, Z. & Kirschner, M. Regulated proteolysis of Xom mediates dorsoventral pattern formation during early Xenopus development. Dev Cell 3, 557-68 (2002).
33. Gao, H., Wu, B., Giese, R. & Zhu, Z. Xom interacts with and stimulates transcriptional activity of LEF1/TCFs: implications for ventral cell fate determination during vertebrate embryogenesis. Cell Res 17, 345-56 (2007).
34. Gao, H. et al. Hom-1, a novel lymphoid-enhancing factor/T-cell factor-associated transcription repressor, is a putative tumor suppressor. Cancer Res 70, 202-11 (2010).
35. Wu, X. et al. Hom-1 trans-activates p53 and p16ink4a to regulate cellular senescence. J Biol Chem 286, 12693-701 (2011).
36. Gao, H. et al. Suppression of Homeobox Transcription Factor Hom-1 Promotes Expansion of Human Hematopoietic Stem/Multipotent Progenitor Cells. J Biol Chem 287, 29979-87 (2012).
37. Gao, H., Wu, B., Le, Y. & Zhu, Z. Homeobox protein Hom-1 induces p53-independent apoptosis in cancer cells. Oncotarget (2016).
38. Wu, B., Gao, H., Le, Y., Wu, X. & Zhu, Z. Xom induces proteolysis of beta-catenin through GSK3beta-mediated pathway. FEBS Lett 592, 299-309 (2017).
39. Wang, C. et al. In vitro characterization of the anti-PD-1 antibody nivolumab, BMS-936558, and in vivo toxicology in non-human primates. Cancer Immunol Res 2, 846-56 (2014).
40. Germeau, C. et al. High frequency of antitumor T cells in the blood of melanoma patients before and after vaccination with tumor antigens. J Exp Med 201, 241-8 (2005).
41. Kamada, N. et al. Unique CD14 intestinal macrophages contribute to the pathogenesis of Crohn disease via IL-23/IFN-gamma axis. J Clin Invest 118, 2269-80 (2008).
42. Rogler, G. et al. Isolation and phenotypic characterization of colonic macrophages. Clin Exp Immunol 112, 205-15 (1998).
43. Eruslanov, E. B. et al. Tumor-associated neutrophils stimulate T cell responses in early-stage human lung cancer. J Clin Invest 124, 5466-80 (2014).
44. Mittal, V. K., Bhullar, J. S. & Jayant, K. Animal models of human colorectal cancer: Current status, uses and limitations. World J Gastroenterol 21, 11854-61 (2015).
45. Wong, H. H. & Chu, P. Immunohistochemical features of the gastrointestinal tract tumors. J Gastrointest Oncol 3, 262-84 (2012).
46. Peters, W. P. et al. Prospective, randomized comparison of high-dose chemotherapy with stem-cell support versus intermediate-dose chemotherapy after surgery and adjuvant chemotherapy in women with high-risk primary breast cancer: a report of CALGB 9082, SWOG 9114, and NCIC MA-13. J Clin Oncol 23, 2191-200 (2005).
47. Opzoomer, J. W., Sosnowska, D., Anstee, J. E., Spicer, J. F. & Arnold, J. N. Cytotoxic Chemotherapy as an Immune Stimulus: A Molecular Perspective on Turning Up the Immunological Heat on Cancer. Front Immunol 10, 1654 (2019).
48. Powley, I. R. et al. Patient-derived explants (PDEs) as a powerful preclinical platform for anti-cancer drug and biomarker discovery. Br J Cancer 122, 735-744 (2020).
49. Noy, R. & Pollard, J. W. Tumor-associated macrophages: from mechanisms to therapy. Immunity 41, 49-61 (2014).
50. De Palma, M. & Lewis, C. E. Macrophage regulation of tumor responses to anticancer therapies. Cancer Cell 23, 277-86 (2013).

51. Galluzzi, L., Buque, A., Kepp, O., Zitvogel, L. & Kroemer, G. Immunological Effects of Conventional Chemotherapy and Targeted Anticancer Agents. Cancer Cell 28, 690-714 (2015).
52. Bracci, L., Schiavoni, G., Sistigu, A. & Belardelli, F. Immune-based mechanisms of cytotoxic chemotherapy: implications for the design of novel and rationale-based combined treatments against cancer. Cell Death Differ 21, 15-25 (2014).
53. Solimini, N. L., Luo, J. & Elledge, S. J. Non-oncogene addiction and the stress phenotype of cancer cells. Cell 130, 986-8 (2007).
54. Mantovani, A., Polentarutti, N., Luini, W., Peri, G. & Spreafico, F. Role of host defense merchanisms in the antitumor activity of adriamycin and daunomycin in mice. J Natl Cancer Inst 63, 61-6 (1979).
55. Ben-Neriah, Y. & Karin, M. Inflammation meets cancer, with NF-kappaB as the matchmaker. Nat Immunol 12, 715-23 (2011).
56. Biswas, S. K. & Lewis, C. E. NF-kappaB as a central regulator of macrophage function in tumors. J Leukoc Biol 88, 877-84 (2010).
57. Arlt, A. et al. Inhibition of NF-kappaB sensitizes human pancreatic carcinoma cells to apoptosis induced by etoposide (VP16) or doxorubicin. Oncogene 20, 859-68 (2001).
58. Wang, S. et al. Activation of nuclear factor-kappaB during doxorubicin-induced apoptosis in endothelial cells and myocytes is pro-apoptotic: the role of hydrogen peroxide. Biochem J 367, 729-40 (2002).
59. Kroemer, G., Galluzzi, L., Kepp, O. & Zitvogel, L. Immunogenic cell death in cancer therapy. Annu Rev Immunol 31, 51-72 (2013).
60. Freeman, A. E. & Hoffman, R. M. In vivo-like growth of human tumors in vitro. Proc Natl Acad Sci U S A 83, 2694-8 (1986).
61. Vescio, R. A., Connors, K. M., Kubota, T. & Hoffman, R. M. Correlation of histology and drug response of human tumors grown in native-state three-dimensional histoculture and in nude mice. Proc Natl Acad Sci U S A 88, 5163-6 (1991).
62. Yoshimasu, T. et al. Data acquisition for the histoculture drug response assay in lung cancer. J Thorac Cardiovasc Surg 133, 303-8 (2007).
63. Yoshimasu, T. et al. In vitro evaluation of dose-response curve for paclitaxel in breast cancer. Breast Cancer 14, 401-5 (2007).
64. Hayashi, Y. et al. Class III beta-tubulin expression in tumor cells is correlated with resistance to docetaxel in patients with completely resected non-small-cell lung cancer. Intern Med 48, 203-8 (2009).
65. Ma, Y. et al. Anticancer chemotherapy-induced intratumoral recruitment and differentiation of antigen-presenting cells. Immunity 38, 729-41 (2013).
66. Correale, P. et al. Dendritic cell-mediated cross-presentation of antigens derived from colon carcinoma cells exposed to a highly cytotoxic multidrug regimen with gemcitabine, oxaliplatin, 5-fluorouracil, and leucovorin, elicits a powerful human antigen-specific CTL response with antitumor activity in vitro. J Immunol 175, 820-8 (2005).
67. Carey, C. D. et al. Topological analysis reveals a PD-L1-associated microenvironmental niche for Reed-Sternberg cells in Hodgkin lymphoma. Blood 130, 2420-2430 (2017).

1. An ex vivo culture model or en bloc culture of solid tumor/cancer (such as lung cancer including NSCLC), for testing the efficacy a therapeutic method on treating the tumor/cancer, comprising freshly isolated tissue sample of the solid tumor/cancer cultured in a suitable mammalian tissue culture medium, wherein the freshly isolated tissue sample is reduced in size to about 1-10 mm (e.g., 2-8 mm, 3-6 mm, about 5 mm) in the largest dimension.

2-5. (canceled)

6. A method to assess the efficacy or effectiveness of a therapy in order to treat a solid tumor/cancer in a subject having said solid tumor/cancer, the method comprising contacting a therapeutic agent for the therapy with the ex vivo culture model or en bloc culture of claim 1, for said solid tumor/cancer isolated from said subject, and identifying a favorable outcome after a sufficient period of time, wherein the favorable outcome indicates that said subject is suitable to be treated by said therapy, and/or wherein the method further comprises selecting said subject for treatment by said therapy upon observation of the favorable outcome, wherein the favorable outcome:
(1) with respect to the therapeutic agent that comprises a chemotherapy agent (such as the chemotherapeutic agent at a sub-therapeutic dose insufficient to treat said solid tumor/cancer), comprises elevated/increased Hom-1 expression in tumor-associated macrophages (TAM) in said ex vivo culture model or en bloc culture (as compared to Hom-1 expression without contacting the therapeutic agent); or,
(2) with respect to the therapeutic agent that comprises an immune checkpoint inhibitor (ICI), comprises cytocidal effect on tumor/cancer cells; activation of cytotoxic T cells (CTL) or CD8⁺ T cells; elevated expression and/or secretion of pro-inflammatory cytokines (such as IL-1β, IL-8, IL-12B and TNF-α) and/or reduction in expression of immune suppressive cytokines (such as IL-4, IL-10, IL13 and TGF-β), and/or death of tumor cells in the tissue cultures.

7. The method of claim 6, wherein the solid tumor/cancer is lung cancer, such as NSCLC, or colorectal cancer.

8. The method of claim 6, wherein the therapy is chemotherapy, immunotherapy, radiation therapy, or a combination thereof.

9. (canceled)

10. The method of claim 6, wherein the ICI comprises an antibody or antigen-binding fragment thereof.

11. The method of claim 10, wherein the antibody or antigen-binding fragment thereof is specific for an inhibitory immune checkpoint target, such as PD-1, PD-L1, PD-L2, CTLA-4/CD152, A2AR, B7-H3/CD276, B7-H4/VTCN1, BTLA/CD272, IDO, KIR, LAG3, NOX2, TIM-3, VISTA, galectin-9, SIGLEC7/CD328, or SIGLEC9.

12-13. (canceled)

14. The method of claim 6, wherein the ex vivo culture model or en bloc culture of said solid tumor/cancer is contacted by said therapeutic agent for at least 1-2 days.

15. The method of claim 6, wherein the method further comprises contacting the ex vivo culture model or en bloc culture of said solid tumor/cancer with a second therapeutic agent.

16. The method of claim 15, wherein the second therapeutic agent comprises a macrophage or a monocyte having elevated/increased Hom-1 expression (e.g., induced or modified to express Hom-1).

17. (canceled)

18. The method of claim 16, wherein the macrophage or a monocyte is induced to express Hom-1 in vitro by contacting the macrophage or monocyte with an agent that induces the expression of Hom-1 or introducing into the macrophage or monocyte a heterologous construct encoding Hom-1.

19. The method of claim 18, wherein the heterologous construct encoding Hom-1 comprises a plasmid encoding Hom-1, an mRNA encoding Hom-1, or a nanoparticle encompassing aa plasmid or an mRNA encoding Hom-1.

20-21. (canceled)

22. The method of claim 6, wherein the sufficient period of time comprises about 3-6 days, such as 3, 4, 5, 6, 7, or 8 days culturing at 37° C. and under 5% CO₂.

23. The method of claim 6, wherein the outcome is determined by isolating single cells from the ex vivo culture model or en bloc culture of said solid tumor/cancer.

24. The method of claim 6, wherein determining the favorable outcome comprises: assessing the viability or death of cancer cells, assessing the number and/or function of CD8⁺ and/or CD4⁺ lymphocytes (optionally including the number of T_{reg}) and/or numbers/functions of macrophages (including TAMs) in the ex vivo culture model or en bloc culture, the expression of cell surface check point inhibitors (such as PD-1 and CTLA-4), the expression of effector molecules (such as the IFN-γ and Granzyme B), the M1-or M2-like phenotype of the TAMs, the expression of immune suppressive cytokines (such as the IL-4, IL-10, IL13 and TGF-β), and/or the expression of the pro-inflammatory cytokines (such as IL-1β, IL-8, IL-12B and TNF-α).

25. The method of claim 6, wherein determining the favorable outcome comprises FACS analysis of isolated single cells from the ex vivo culture model or en bloc culture, and/or ELISA analysis of culture supernatants from the ex vivo culture model or en bloc culture (e.g., ELISA analysis of IL-2 and IFN-γ expression).

26. The method of claim 6, wherein determining the favorable outcome comprises determining cell surface expression of CD68 and CD206 on TAMs (e.g., via FACS), and/or Hom-1 expression level (e.g., via qRT-PCR analysis), determining the percentage of T_{reg} cells (e.g., by FACS analysis of the percentage changes of the CD4⁺CD25⁺FoxP3⁺ cells), and/or determining the change or enhancement of CD8⁺ T cell activation upon contact by the therapeutic agent (e.g., an ICI antibody, such as anti-PD-1 antibody).

27-28. (canceled)

29. The method of claim 6, wherein the method comprises comparing the favorable outcome with that of a control outcome obtained by contacting a control therapeutic agent for the therapy with the ex vivo culture model or en bloc culture of said solid tumor/cancer.

30. (canceled)

31. A method of treating a cancer, such as a solid cancer/tumor (e.g., lung cancer including NSCLC), the method comprising administering a therapy to a subject having said cancer, wherein the subject has been validated to respond to treatment by said therapy according to a favorable outcome in the method of claim 6 to assess the efficacy or effectiveness of the therapy for treating said solid tumor/cancer using the ex vivo culture model or en bloc culture of said solid tumor/cancer.

32-44. (canceled)

45. A method to enhance immune checkpoint inhibitor (ICI)-mediated therapy or chemotherapy of a cancer (e.g., treatment of NSCLC) in a subject, the method comprising promoting tumor-specific activation of CD8⁺ T cells in tumor microenvironment (TME) of the cancer through Hom-1 activation in, or Hom-1 mediated activation of, tumor-associated macrophages (TAMs).

46-55. (canceled)

56. A method to select an effective dosage of a therapy (e.g., chemotherapeutic agent, targeted therapy, immunotherapeutic treatment, radiation therapy, or combinations thereof) for treating a cancer (e.g., lung cancer or colorectal cancer) in a subject, the method comprising determining Hom-1 activation in, or Hom-1 mediated activation of, tumor-associated macrophages (TAMs) of said cancer (e.g., ex-vivo, in vivo, or both), upon contacting the cancer with said therapy, wherein the minimum effective dosage of said therapy that leads to Hom-1 activation, or a higher dosage, is selected to be the effective dosage.

57-58. (canceled)
